(19) United States
(12) Patent Application Publication
(10) Pub. No.: US 2025/0221845 A1
(43) Pub. Date: Jul. 10, 2025

(54) MICRO-CONTROL PORTABLE URINAL

(71) Applicants: Gary CHIU, Richardson, TX (US); Joanne CHAO, Richardson, TX (US); Hwai-Luh CHANG, Richardson, TX (US)

(72) Inventors: Gary CHIU, Richardson, TX (US); Joanne CHAO, Richardson, TX (US); Hwai-Luh CHANG, Richardson, TX (US)

(21) Appl. No.: 18/408,625

(22) Filed: Jan. 10, 2024

(51) Int. Cl. A61F 5/453

(52) U.S. Cl. CPC A61F 5/453

(57) ABSTRACT

The portable urinal has a urine collector, a suction assembly, and a container. The urine collector has a first opening, a second opening, an inner space, and a urine level sensor. The inner space is formed in the urine collector. The urine level sensor is in the inner space to detect a urine level. The suction assembly has a urine pump, a urine exporting tube, and a controlling unit. The urine pump is adjustable in suctioning power and fluidly communicates with the urine collector. The urine exporting tube is connected to the urine pump and fluidly communicates with a urine reservoir of the container. The controlling unit is communicatively connected to the urine pump and the urine level sensor. The portable urinal is capable of adjusting the suctioning power of the urine suction pump by the amount of the urine in the urine collector.

### BACKGROUND

### 1. Field of the Invention

The present invention relates to a portable urinal, especially to a portable urinal that is capable of actively suctioning the urine in the urine collector.

### 2. Description of the Prior Arts

A urine collection device is a device that helps people who are restricted from access to toilet facilities, such as on a long-distance drive or being bedridden. The conventional urine collection device includes a urine collector and a urine container, and a tube connected to the urine collector and the urine container; a user urinates to the urine collector, and then urine flows into the urine container through the tube by gravity.

However, due to flowing passively by gravity, once an exporting rate of urine in the urine collector is less than a urinating rate of the user, the urine may overflow from the urine collector and the urine collector may be contaminated by precipitates of the urine, causing odors or infections to the user.

In order to solve the aforementioned issue, an electrical suction unit is introduced into the urine collection device. The electrical suction unit is capable of actively suctioning the urine in the urine collector, but the electrical suction unit would keep suctioning without adjusting power by the amount of the urine in the urine collector, and thus the electrical suction unit keeps consuming electricity and generating noise.

Furthermore, the urine container is usually a urine bag which has to be set below the genitals and fixed on the leg, such that moves of the user are restricted; also, the urine in the urine bag may flow back once the urine bag is compressed since the urine bag is made of soft materials. Besides, the conventional urine container does not have the capacity of alerting the user when the conventional urine container is full, which may lead to the urine overflowing.

Some users may resort to diapers, but the used diapers need to be replaced frequently to prevent odors, skin irritation, and leakage, and thus produces a large amount of garbage and costs much; also, the diapers are hard to be packed and carried due to large space occupation.

To overcome the shortcomings, the present invention provides a portable urinal to mitigate or obviate the aforementioned problems.

### SUMMARY

The main objective of the present invention is to provide a portable urinal that is capable of adjusting suction power according to an amount of urine in a urine collector.

A portable urinal has a urine collector, a suction assembly, and a container. The urine collector is tubular in shape, and has a top end and a bottom end opposite to each other, and the bottom end is closed. The urine collector has a first opening, a second opening, an inner space, and a urine level sensor. The first opening and the second opening are formed on the top end of the urine collector. The inner space is formed in the urine collector and fluidly communicates with the first opening and the second opening. The urine level sensor is disposed in the inner space and adjacent to the bottom end of the urine collector, and the urine level sensor is configured to detect a urine level in the inner space. The suction assembly has a urine pump, a urine exporting tube, and a controlling unit. The urine pump is adjustable in suctioning power and fluidly communicates with the inner space via the second opening of the urine collector. The urine exporting tube is connected to the urine pump. The controlling unit is communicatively connected to the urine pump and the urine level sensor. The container has a urine opening and a urine reservoir. The urine opening fluidly communicates with the urine reservoir, and the urine exporting tube fluidly communicates with the urine reservoir.

Therefore, the portable urinal is capable of increasing the suctioning power of the urine suction pump as long as an amount of the urine in the urine collector increases, and decreasing the suctioning power of the urine suction pump as long as the amount of the urine decreases, thereby synchronously exporting the urine during urinating in a comfortable manner. Furthermore, due to actively suctioning the urine in the urine collector, the user does not need to keep the container located below the genitals for gravity, and the overflowing of the urine from the urine collector is also prevented.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a portable urinal in accordance with the present invention;

FIG. 2 is an exploded view of the portable urinal in FIG. 1;

FIG. 3 is a perspective view of a urine collector of the portable urinal in FIG. 1;

FIG. 4 is an exploded view of the urine collector in FIG. 3;

FIG. 5 is a cross-sectional view of the urine collector in FIG. 3;

FIGS. 6 and 7 are cross-sectional views of the urine collector in FIG. 3, viewed from another angle and showing different urine levels;

FIG. 8 is a cross-sectional view of a container of the portable urinal in FIG. 1; and

FIG. 9 is an enlarged view of the suction assembly in FIG. 1, showing the structure inside a case.

### DETAILED DESCRIPTION

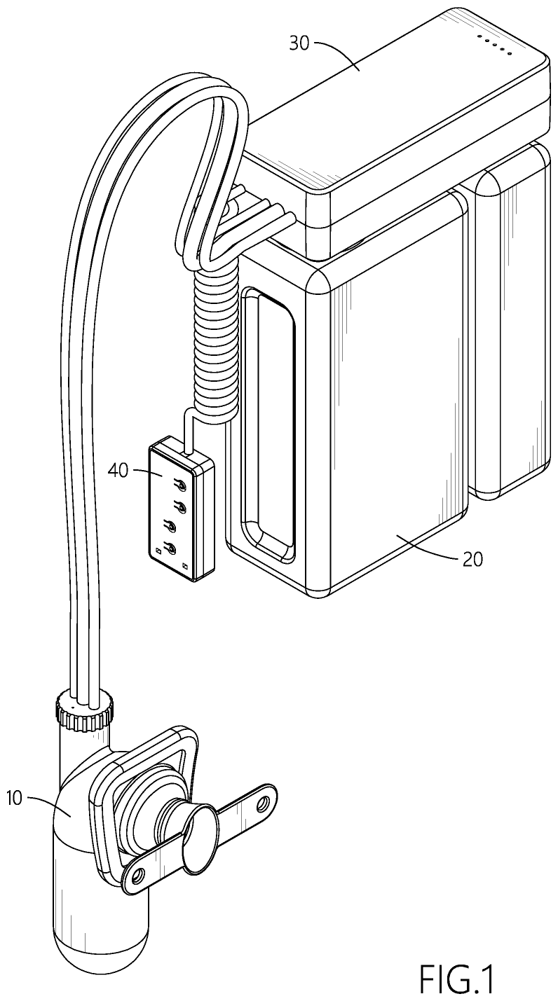

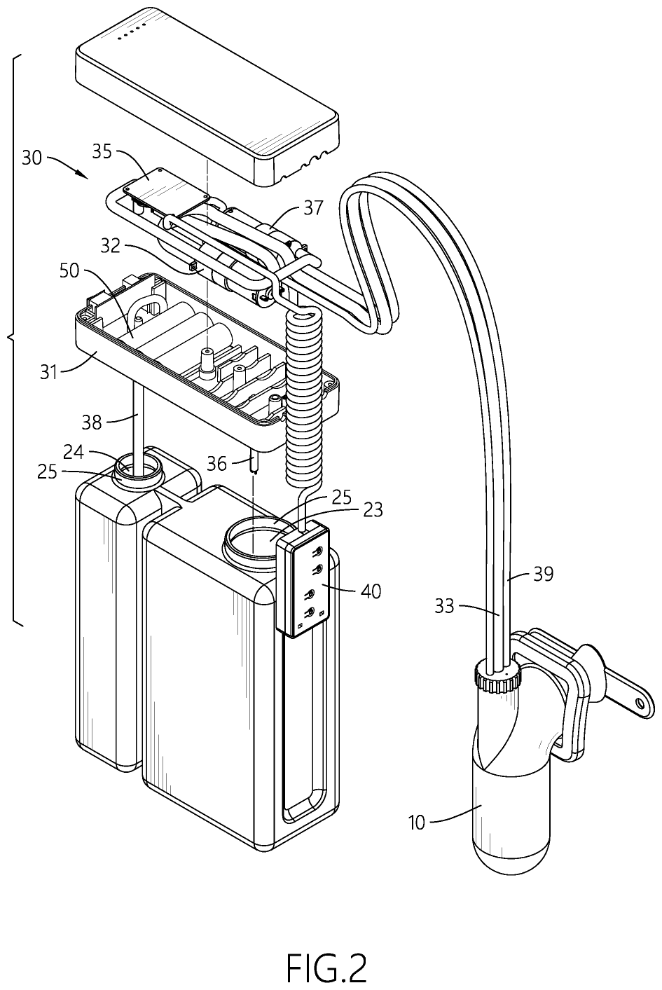

With reference to FIGS. 1 and 2, a portable urinal in accordance with the present invention has a urine collector 10, a container 20, and a suction assembly 30.

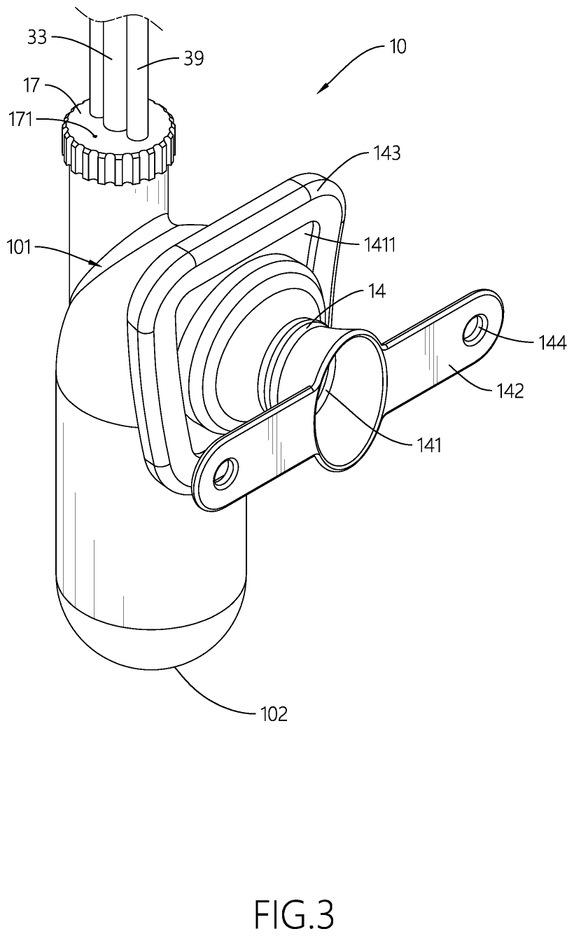

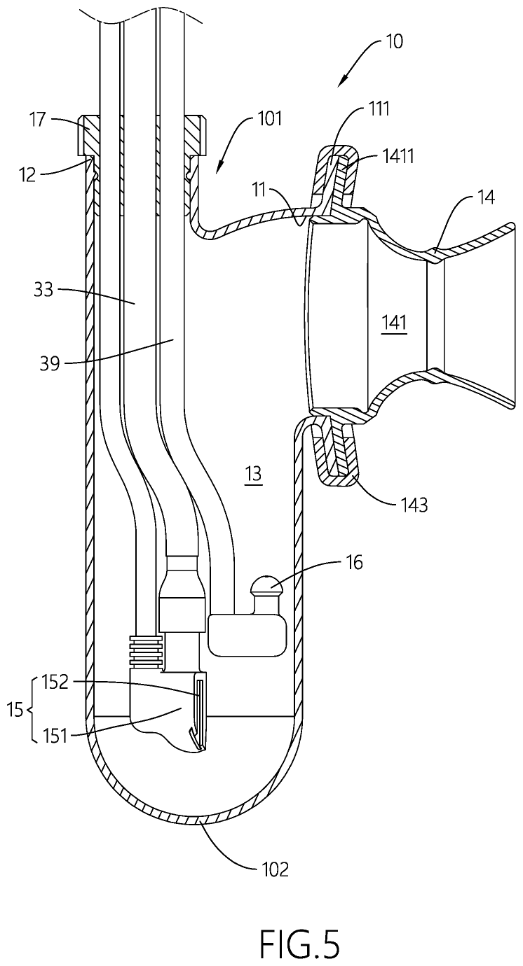

With reference to FIGS. 3 to 5 together, the urine collector 10 is tubular in shape and has a top end 101 and a bottom end 102 which are opposite to each other. The bottom end 102 is closed. The urine collector 10 has a first opening 11, a second opening 12, an inner space 13, a retainer 14, a urine level sensor 15, a nozzle 16, and a pipe holder 17.

In this embodiment, the first opening 11 is oblique with respect to an axial direction of the urine collector 10, thereby being easy for a user to insert a genital into the urine collector 10 to urinate, but it is not limited thereto. The inner space 13 is formed in the urine collector and fluidly communicates with the first opening 11 and the second opening 12.

The retainer 14 is tubular in shape, and an end of the retainer 14 is connected to the first opening 11. The retainer 14 is elastic in this embodiment, preferably, the retainer 14 is made of silicone, but it is not limited thereto.

The retainer 14 has a channel 141 and a handle 142, and the handle 142 is connected to another end of the retainer 14. The channel 141 is formed through the two opposite ends of the retainer 14, and the channel 141 is shaped narrow in middle and wide at two ends of the channel 141 to help fix the genitals of the user. Furthermore, in this embodiment, the retainer 14 has two of the handles 142 for the user to hold the urine collector 10 and each one of the handles 142 is strip-like. Besides, the retainer 14 may be made of other materials which are bendable and would not irritate skin.

Moreover, in this embodiment, the retainer 14 and the first opening 11 are connected with a securing clip 143. To be more precise, a first securing portion 111 is connected to the urine collector 10 and surrounds the first opening 11; likely, a second securing portion 1411 is connected to the retainer 14 and surrounds an opening of the channel 141 oriented toward the first opening 11. The securing clip 143 clips the first securing portion 111 and the second securing portion 1411 together to fix the retainer 14 at the first opening 11 of the urine collector 10, but it is not limited thereto, as a configuration of how the retainer 14 is fixed on the urine collector 10 may be altered according to need.

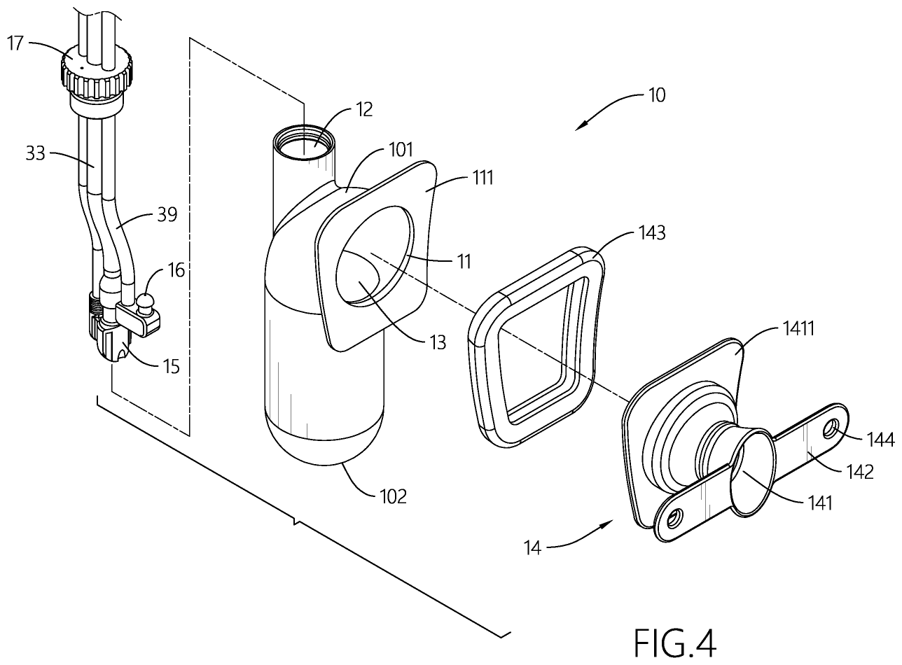

With reference to FIGS. 4 to 7, the urine level sensor 15 is disposed in the inner space 13 and adjacent to the bottom end 102 of the urine collector 10. The urine level sensor 15 is configured to detect a urine level in the inner space 13. To be more precise, the urine level sensor 15 is connected to a cable which is mounted through the second opening 12, and thereby the urine level sensor 15 is hung in the inner space 13.

The urine level sensor 15 has a base 151 and a sensor portion 152. The sensor portion 152 is mounted on the base 151. The sensor portion 152 is linear and extends along an axial direction of the inner space 13 of the urine collector 10, and thus once a urine level in the urine collector 10 has changed, a crossing position of said urine level with respect to the sensor portion 152 would change as well. The sensor portion 152 in this embodiment includes two electrodes spaced apart from each other, and since the urine is conductive, the two electrodes are capable of detecting the voltage of the urine.

The nozzle 16 is disposed in the inner space 13 of the urine collector 10, likely, the nozzle 16 is hung in the inner space 13 of the urine collector 10 by a tube, but it is not limited thereto.

With reference to FIGS. 3 to 5, the pipe holder 17 is mounted at the second opening 12, and the cable connected to the urine level sensor 15 and the tube connected the nozzle 16 pass the second opening 12 via the pipe holder, and thereby the urine level sensor 15 and the nozzle 16 are hung in the inner space 13, but it is not limited thereto. In addition, in this embodiment, the pipe holder 17 is sealed at the second opening 12 and has a pressure balancing hole 171. The pressure balancing hole 171 fluidly communicates with the inner space 13 and an exterior of the urine collector 10.

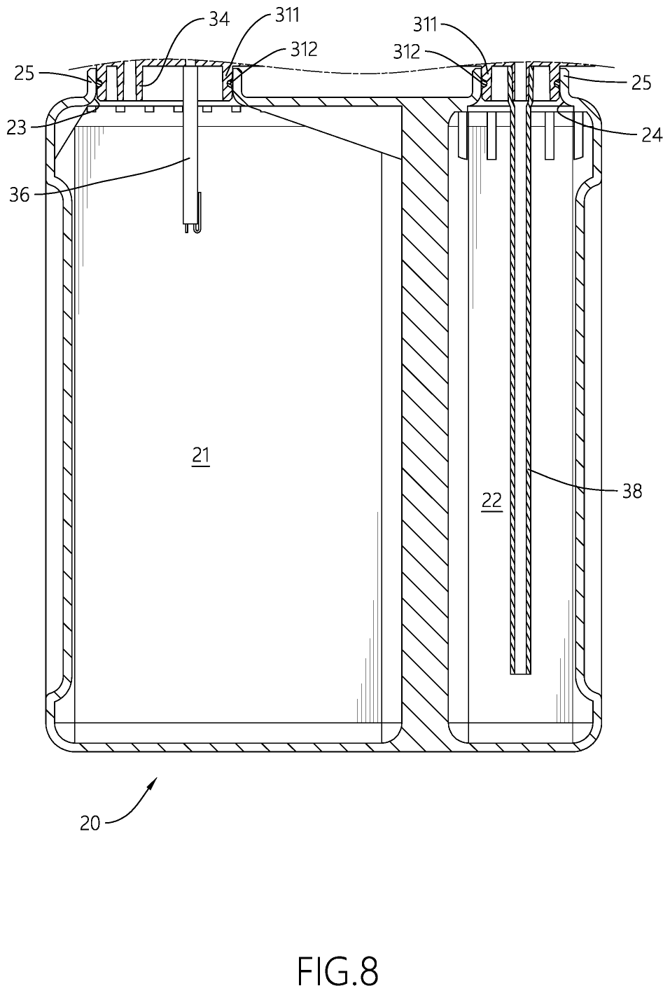

With reference to FIGS. 1, 2 and 8, the container 20 has a urine reservoir 21 and a water reservoir 22, and the urine reservoir 21 and the water reservoir 22 are isolated from each other. A urine opening 23 fluidly communicates with the urine reservoir 21, and a water opening 24 fluidly communicates with the water reservoir 22.

Besides, in this embodiment, two connecting portions 25 are mounted on the container 20 and respectively surround the urine opening 23 and the water opening 24, and each one of the connecting portions 25 protrudes outward of the container 20, but it is not limited thereto.

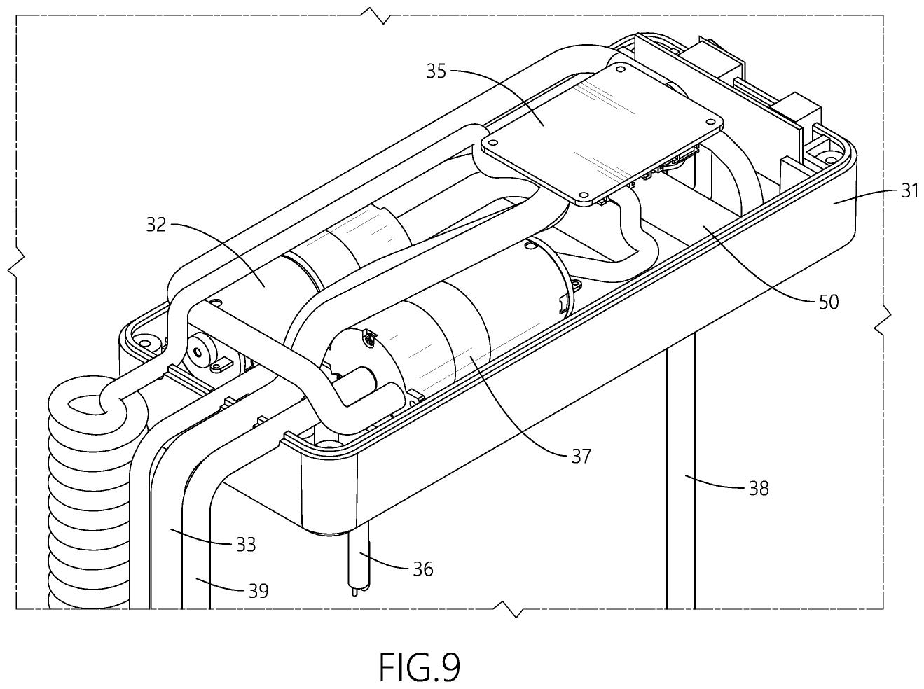

With reference to FIGS. 2, 5, 8, and 9, the suction assembly 30 has a case 31, a urine pump 32, a urine suction tube 33, a urine exporting tube 34, a controlling unit 35, a urine storage sensor 36, a water pump 37, a water suction tube 38, and a water exporting tube 39. The urine pump 32, the water pump 37, and the controlling unit 35 are disposed in the case 31, but it is not limited thereto, as the suction assembly 30 may not have the case 31.

The case 31 has two docking portions 311 protruding outward of the case 31, and the two docking portions 311 are detachably connected to the two connecting portions 25 of the container 20 respectively. To be more precise, in this embodiment, the docking portions 311 and the connecting portions 25 are annular walls; each one of the docking portions 311 has an outer annular surface, and each one of the connecting portions 25 has an inner annular surface, and each one of said outer annular surfaces detachably contacts a corresponding one of said inner annular surfaces. Besides, in this embodiment, the case 31 may further have two sealing rings 312 respectively mounted on the two docking portions 311, but it is not limited thereto.

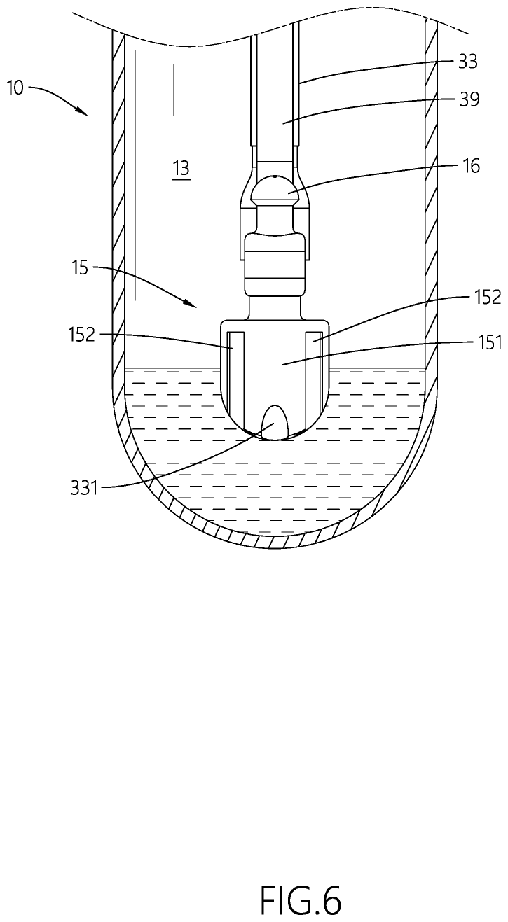

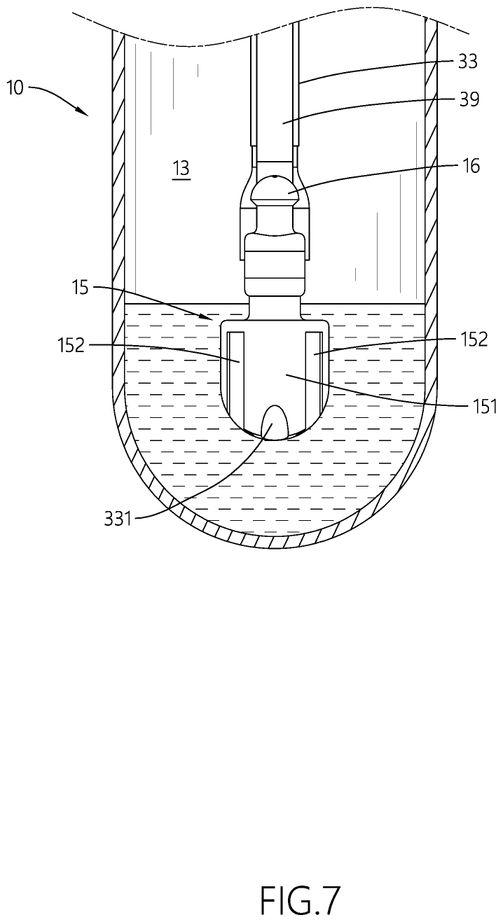

The urine pump 32 is adjustable in a suctioning power. The urine suction tube 33 is connected to the urine pump 32 and the urine level sensor 15, and the urine suction tube 33 extends into the inner space 13 of the urine collector 10 via the second opening 12 and is fixed by the pipe holder 17. To be more precise, as shown in FIGS. 6 and 7, a urine suction opening 331 is formed on the base 151 of the urine level sensor 15, and the urine suction tube 33 fluidly communicates with the urine suction opening 331 and the urine pump 32, and thereby the urine in the inner space 13 is suctioned into the urine suction tube 33 via the urine suction opening 331, but it is not limited thereto.

The urine exporting tube 34 is connected to the urine pump 32, and fluidly communicates with the container 20; to be more precise, the urine exporting tube 34 passes through the case 31 and one of the docking portions 311 to fluidly communicate with the urine reservoir 21 of the container 20.

The controlling unit 35 is communicatively connected to the urine pump 32 and the urine level sensor 15; in this embodiment, the controlling unit 35 is electronically connected to the urine pump 32 and the urine level sensor with cables, but it is not limited thereto, as the controlling unit 35 may wirelessly communicate with the urine pump 32 and the urine level sensor 15. The controlling unit 35 is capable of adjusting a suction power of the urine pump 32 according to the urine level in the inner space 13 of the urine collector 10.

The urine storage sensor 36 is located at the urine opening 23 of the container 20, and the urine storage sensor 36 is communicatively connected to the controlling unit 35; to be more precise, the urine storage sensor 36 passes through one of the docking portions 311 and extends to the urine reservoir 21 of the container 20. Likely, the urine storage sensor 36 is connected to the controlling unit 35 with a cable in this embodiment, but it is not limited thereto; the portable urinal may not have the urine storage sensor 36.

The water suction tube 38 passes through the case 31 and another one of the docking portions 311, and the water suction tube 38 extends to and fluidly communicates with the water reservoir 22 of the container 20; to be more precise, an end of the water suction tube 38 is disposed in the water reservoir 22 of the container 20, and another end of the water suction tube 38 is connected to the water pump 37.

Two ends of the water exporting tube 39 are respectively connected to the water pump 37 and the nozzle 16, and thereby the water pump 37 is capable of exporting water to the nozzle 16. The water exporting tube 39 extends into the inner space 13 of the urine collector 10 via the second opening 12, and the water exporting tube 39 is mounted through the pipe holder 17, and thereby the nozzle 16 is hung in the inner space 13 by the water exporting tube 39.

The controlling panel 40 is communicatively connected to the controlling unit 35. In this embodiment, the controlling panel 40 is connected to the controlling unit 35 with a cable, but it is not limited thereto; furthermore, the controlling panel 40 further has a vibrator and an LED light (not shown in the figures) that are configured to remind the user if the urine reservoir 21 of the container 20 is full.

In this embodiment, the suction assembly 30 may further have a battery module 50, and the battery module 50 is mounted in the case 31 and supplies electricity to the controlling unit 35, the urine pump 32, and the water pump 37; besides, the battery module 50 may be rechargeable, but it is not limited thereto.

The functions of the portable urinal in this embodiment are demonstrated with a male user, but it is not limited thereto, as the portable urinal of this disclosure is applicable to female users as well.

The user urinates to the urine collector 10, and when the urine level rises in the inner space 13, the controlling unit 35 increases the suctioning power of the urine pump 32 to increase an exporting rate of the urine in the urine collector 10; on the other hand, as long as the urine level lowers down, the controlling unit 35 would reduce the suctioning power of the urine pump 32, and thus the portable urinal is capable of preventing pulling or tearing the urination organ of the user which makes the user uncomfortable.

As shown in FIGS. 6 and 7, since the urine is conductive, when the urine contacts the sensor portion 152 of the urine level sensor 15, the controlling unit 35 would receive an electrical signal; furthermore, because the sensor portion 152 is linear and extends along the axial direction of the inner space 13, once the crossing position of the liquid level of the urine and the sensor portion 152, i.e., an area of the sensor portion 152 soaked in the urine, has changed, a voltage change would be detected as an analog signal, and then the controlling unit 35 would adjust the power of the urine pump 32 according to the signal as mentioned above.

Therefore, the portable urinal in this disclosure is capable of activating or inactivating the urine pump 32, or adjusting the power of the urine pump 32 to synchronously export the urine when the user is urinating, thereby preventing the urine overflowing from the urine collector 10. Besides, by actively suctioning the urine with the suction assembly 30, the user does not need to keep the container 20 located below the genitals or fixed on a leg, which allows the user to move normally.

For those users who are restricted from standing or sitting normally, the retainer 14 helps fix the urine collector 10 on the user. To be more precise, the user can insert the genitals into the channel 141, and the retainer 14 is fixed on the genitals since the channel 141 is shaped narrow in middle. Besides, each one of the handles 142 may form a connecting hole 144, and the user may pass a string (not shown in the drawings) through the connecting holes 144 to help fix the urine connector 10 on the genitals, but it is not limited thereto.

Furthermore, the portable urinal in this embodiment is capable of washing the urine collector 10 and even the genitals of the user. To be more precise, the wash function may be triggered automatically or manually by the user with the controlling panel 40, and after the wash function is activated, the water pump 37 pumps out water from the water reservoir 22 of the container 20, and then the nozzle 16 in the urine collector 10 sprays the water to wash the inner wall surface 101 of the urine collector 10 to prevent urine residues, or wash the genitals of the user.

Besides, the portable urinal is capable of reminding the user if the urine reservoir 21 is full; to be more precise, the urine storage sensor 36 located at the urine opening 23 of the container 20 is capable of detecting the amount of the urine in the urine reservoir 21, and the controlling unit 35 may alert the user with the vibrator and/or the LED light of the controlling panel 40 when the urine reservoir 21 is full.

In summary, the portable urinal in this disclosure actively suctions the urine in the urine collector 10 instead of flowing passively by gravity, thereby preventing the urine overflowing or residues in the urine collector 10, and furthermore, the portable urinal is capable of adjusting the suctioning power of the urine pump 32 according to the urine in the urine collector 10; besides, the portable urinal is also capable of washing the genitals of the user and reminding the user if the urine reservoir 21 of the container 20 is full, with the aforementioned functions, the portable urinal in this disclosure helps prevent odor and infections, exports the urine in the urine collector 10 in a comfortable manner, and allows the user to urinate without limiting body movements.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A portable urinal comprising:
a urine collector being tubular in shape, and the urine collector having:
a top end and a bottom end opposite to each other, and the bottom end being closed;
a first opening and a second opening formed on the top end of the urine collector;
an inner space formed in the urine collector and fluidly communicating with the first opening and the second opening; and
a urine level sensor disposed in the inner space and adjacent to the bottom end of the urine collector, and the urine level sensor configured to detect a urine level in the inner space;
a suction assembly having:
a urine pump being adjustable in suctioning power and fluidly communicating with the inner space via the second opening of the urine collector;
a urine exporting tube connected to the urine pump;
a controlling unit communicatively connected to the urine pump and the urine level sensor;
a container having:
a urine opening and a urine reservoir, and the urine opening fluidly communicating with the urine reservoir, the urine exporting tube fluidly communicating with the urine reservoir.

2. The portable urinal as claimed in claim 1, wherein the urine level sensor has:
a sensor portion being linear and extending along an axial direction of the inner space of the urine collector.

3. The portable urinal as claimed in claim 1, wherein:
a urine suction opening is formed on the urine level sensor; and
the suction assembly has:
a urine suction tube fluidly communicating with the urine pump and the urine suction opening.

4. The portable urinal as claimed in claim 2, wherein:
a urine suction opening is formed on the urine level sensor; and
the suction assembly has:
a urine suction tube fluidly communicating with the urine pump and the urine suction opening.

5. The portable urinal as claimed in claim 1, wherein the suction assembly further has:
a urine storage sensor located at the urine opening of the container, and the urine storage sensor communicatively connected to the controlling unit.

6. The portable urinal as claimed in claim 4, wherein the suction assembly further has:
a urine storage sensor located at the urine opening of the container, and the urine storage sensor communicatively connected to the controlling unit.

7. The portable urinal as claimed in claim 1, wherein:
the container further has:
a water opening and a water reservoir, and the water opening fluidly communicating with the water reservoir, the water reservoir and the urine reservoir isolated from each other;
the urine collector further has:
a nozzle disposed in the inner space of the urine collector;
the suction assembly further has:
a water pump fluidly communicating with the nozzle; and
a water suction tube, an end of the water suction tube disposed in the water reservoir, and another end of the water suction tube connected to the water pump.

8. The portable urinal as claimed in claim 6, wherein:
the container further has:
a water opening and a water reservoir, and the water opening fluidly communicating with the water reservoir, the water reservoir and the urine reservoir isolated from each other;
the urine collector further has:
a nozzle disposed in the inner space of the urine collector;
the suction assembly further has:
a water pump fluidly communicating with the nozzle; and
a water suction tube, an end of the water suction tube disposed in the water reservoir, and another end of the water suction tube connected to the water pump.

9. The portable urinal as claimed in claim 1, wherein the urine collector further has:
a retainer being tubular in shape, and an end of the retainer connected to the first opening.

10. The portable urinal as claimed in claim 8, wherein the urine collector further has:
a retainer being tubular in shape, and an end of the retainer connected to the first opening.

11. The portable urinal as claimed in claim 9, wherein the retainer is elastic and has:
a channel formed through two opposite ends of the retainer, and the channel being narrow in middle and wide at two ends of the channel.

12. The portable urinal as claimed in claim 10, wherein the retainer is elastic and has:
a channel formed through two opposite ends of the retainer, and the channel being narrow in middle and wide at two ends of the channel.

13. The portable urinal as claimed in claim 9, wherein the retainer has:
a handle connected to another end of the retainer.

14. The portable urinal as claimed in claim 12, wherein the retainer has:
a handle connected to another end of the retainer.

15. The portable urinal as claimed in claim 13, wherein the retainer has:
a connecting hole formed through the handle.

16. The portable urinal as claimed in claim 14, wherein the retainer has:
a connecting hole formed through the handle.

17. The portable urinal as claimed in claim 1, wherein the urine collector further has:
a pipe holder mounted at the second opening, and the pipe holder having a pressure balancing hole, the pressure balancing hole fluidly communicating with the inner space and an exterior of the urine collector.

18. The portable urinal as claimed in claim 16, wherein the urine collector further has:
a pipe holder mounted at the second opening, and the pipe holder having a pressure balancing hole, the pressure balancing hole fluidly communicating with the inner space and an exterior of the urine collector.

19. The portable urinal as claimed in claim 1, wherein the portable urinal further has:
a controlling panel communicatively connected to the controlling unit.

20. The portable urinal as claimed in claim 18, wherein the portable urinal further has:
a controlling panel communicatively connected to the controlling unit.
